(19) United States
(12) Patent Application Publication
(10) Pub. No.: US 2008/0275461 A1
(43) Pub. Date: Nov. 6, 2008

(54) Capsular Ring

(76) Inventor: Ravi Nallakrishnan, Westmont, IL (US)

Correspondence Address: LAW OFFICES OF JERRY A. SCHULMAN, 1S376 SUMMIT AVENUE, COURT C, OAKBROOK TERRACE, IL, 60181, US

(21) Appl. No.: 11/744,216

(22) Filed: May 3, 2007

(51) Int. Cl. A61F 9/00

(52) U.S. Cl. 606/107

(57) ABSTRACT

A capsular guide for conducting capsulorhexis in an eye has a guide ring adapted to be retaine against the capsular bag. In a first version the retaining element is at least one haptic. In another version the retaining element is a second ring flexibly attached to the first ring.

### FIELD OF THE INVENTION

The present invention relates generally to the field of ophthalmic surgery and, more particular, to apparatus and methods for performing capsulorrhexis on the lens capsule of a human eye.

### BACKGROUND OF THE INVENTION

Removal of the lens in an eye is required by such physical conditions as cataracts or trauma to the eye. Typically, the removal of a lens is accomplished by making an opening in the anterior wall of the lens capsule and using surgical techniques such as phacoemulsification to fragment and remove the lens. Thereafter, an artificial intraocular lens (IOL) is inserted through the opening to replace the damaged or diseased lens.

To make the opening a surgeon will use instruments such as forceps, needles and the like to puncture the anterior capsular wall, grip the wall at the site of the puncture and tug or tear the wall, preferably in a circular pattern to remove a segment of the wall large enough and appropriately shaped to receive an artificial IOL. This calls for great skill on the part of the surgeon. It is not uncommon to produce a jagged or uneven opening as the wall tears. A smooth edge surrounding the opening is desirable to avoid problems with loose flaps or strips that may be snagged when the lens particles are aspirated after the lens has been emulsified.

The problem of making a clean and well-shaped opening in the anterior wall and attendant attempts to resolve it are well represented in the prior art.

U.S. Pat. No. 5,342,377 (Lazerson) teaches and describes a rotating blade capsulotomy instrument and method of performing a capsulotomy. The instrument described in Lazerson has a pivotable blade used to incise the wall. The blade pivots with respect to the handle enabling the surgeon to maneuver the handle to make a more circular cut.

U.S. Pat. No. 4,530,359 (Healfcott et all) teaches and describes an OPTHALMIC perforating instrument and surgical method employing said instrument. Healfcott et al uses a hollow tube from which a sharpened wire can be extended and used to incise a circular cut on the anterior wall.

U.S. Pat. No. 6,551,326 (Van Heugten et al) teaches and describes a capsulorrhexis device comprising a hollow tube from which a wire may be extended. The wire has a cutting blade at one end and forms a loop as it is extended. As the wire is drawn back into the tube, the wire follows a circular shape and the blade makes the necessary cut.

U.S. Pat. No. 5,860,994 (Yaacobi) teaches and describes a remotely operable intraocular surgical instrument for automated capsulectomies. Yaacobi teaches a handle having an arm protruding therefrom with cutting blade mounted to the arm at a distance from the handle. The arm is rotatably attached to a ratchet wheel which turns the arm in a circular pattern when activated, thereby making the necessary cut.

U.S. Pat. No. 4,766,897 (Smirmaul) teaches and describes a capsulectomy surgical instrument having a handle from which a probe protrudes and a circular cutting device positioned at the end of the tube. As the handle is squeezed, the tube rotates and a cutting edge on the tube makes a circular cut.

U.S. Pat. No. 5,423,841 (Kornefeld) teaches and describes an intraocular knife. Kornefeld has a rotatable blade mounted to a forceps-like handle and the blade is rotated as the handle is squeezed in order to effect a cut through the anterior wall.

U.S. Pat. No. 5,728,117 (Lash) teaches and describes a retractable capsulorrhexis instrument consisting of a tube within which a flexible band is held. The band when forced from the tube assumes a circular shape. The cutting edge is mounted on the band and as the band is forced from the tube, the cutting edge is pushed against the interior wall, a circular cut is made.

Also known in the art are ring-like devices insertable into portions of the eye.

U.S. Pat. No. 6,096,076 (Sylvestrini et al) teaches and describes a hybrid intrastromal corneal ring insertable into the anterior chamber of the eye to correct aberrations in the shape of the cornea.

U.S. Pat. No. 5,645,582 (Sylvestrini et al) teaches and describes an overlapping ring device for corneal curvature adjustment. Another type of corneal ring which, when inserted, is intended to correct the shape of the cornea.

U.S. Pat. No. 6,749,631 (Pietrini et al) teaches and describes a capsular bag ring and an assembly constituted by such a ring and an injector therefore. The device of Petrini et al comprises a ring, which, when allowed to expand within the capsular bag, holds the capsular bag in a circular shape.

U.S. Pat. No. 6,364,906 (Baikoff et al) teaches and describes an intraocular implant with flexible optical part and single circular loop, comprising an artificial IOL to which a single haptic is attached with the haptic extending substantially around and spaced apart from the IOL. The haptic is intended to contact the interior of the capsular bag to hold the IOL in place.

U.S. Pat. No. 5,944,752 (Sylvestrini et al) teaches and describes an astigmatic correcting intrastromal corneal insert insertable into the anterior chamber to correct the shape of the cornea and having a periphery which is irregular in cross-sectional shape.

U.S. Pat. No. 5,405,384 (Sylvestrini) teaches and describes an astigmatic correcting intrastromal cornea ring which functions in much the same fashion as the previously-described Sylvestrini patents.

The foregoing references demonstrate that a significant amount of manual skill and dexterity is still required in order to make a circular or near-circular cut.

Use of instruments having retractable or extendable bands require the surgeon to exert a force on the handle of the instrument in order to move the band while, at the same time, exerting a force against the anterior wall to provide enough pressure to force the cutting edge through the anterior wall to make the cut.

### SUMMARY OF THE INVENTION

The present invention comprises an insertable guide having a ring portion within which capsulorhexis is carried out. A first embodiment of the present invention comprises a hollow ring to which a pair of haptics is attached which, when inserted into the anterior chamber, holds the ring in position on the capsular bag.

A second variation includes upper and lower collars formed of elastomeric material with the lower collar anchoring the guide in the space above the capsular bag and the upper collar acting as the guide within which capsulorhexis is carried out.

A third variation of the foregoing includes an embodiment wherein the lowermost surface of the capsular ring has grippers formed thereon, intended to frictionally engage the capsular bag to keep the ring from moving while capsulorhexis is carried out.

These and further details of the present invention will be better appreciated by considering the accompanying drawings in which:

### BRIEF DESCRIPTION OF THE DRAWINGS

FIG. 1 is a horizontal cross-sectional view of the eye taken from U.S. Pat. No. 6,096,076, illustrating the structure of the eye;

FIG. 2 is an enlarged portion of FIG. 1;

FIG. 3 is a top view of a first embodiment of the present invention with the haptics partially deployed;

FIG. 4 is a view of the embodiment of FIG. 3 with the haptics fully deployed;

FIG. 5 is a horizontal cross-sectional view showing the embodiment of FIG. 3 installed with the haptics deployed in the anterior chamber;

FIG. 6 is a horizontal cross-sectional view showing the embodiment of FIG. 3 installed with the haptics deployed under the iris;

FIG. 7 is a lateral view of a second embodiment of the present invention;

FIG. 8 is a top view of the embodiment of FIG. 7;

FIG. 9 is a horizontal cross-sectional view showing the embodiment of FIG. 7 installed in the anterior chamber;

FIG. 10 is a bottom view of a third embodiment of the present invention, showing grippers formed thereon; and

FIG. 11 is a lateral view of the embodiment shown in FIG. 10.

### DETAILED DESCRIPTION OF THE DRAWINGS

While the following describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

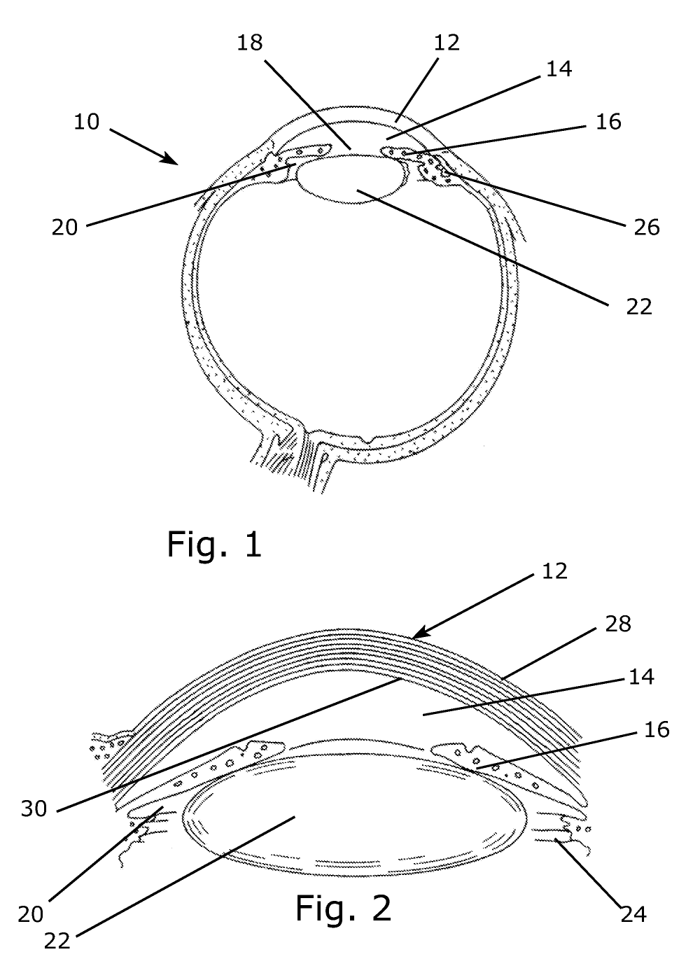

Referring now to FIG. 1, the numeral 10 identifies a horizontal sectional view of a human eye having a cornea 12, an anterior chamber 14, an iris 16 and a pupil 18. Positioned behind iris 16 and in line with pupil 18 is capsular bag 22 within which the crystalline lens is held. A posterior chamber 20 is positioned between iris 16 and capsular bag 22. A ciliary body 26 helps support capsular bag 22.

Referring now to FIG. 2, it can be seen that cornea 12 is made up of a number of individual layers of tissue with the outer layer being the epithelium 28 and the innermost layer being the endothelium 30. A ciliary zonule 24 consists of a series of transparent fibers joining the ciliary body 26 and the capsular bag 22 to hold the capsular bag 22 in position. When performing capsulorhexis, an incision is made through cornea 12 through which typically a forceps is inserted and used to pierce the anterior membrane 32 of capsular bag 22 and to tear it in a circular pattern to allow the damaged or diseased lens to be removed by phacoemulsification and, thereafter, the insertion of an IOL into capsular bag 22.

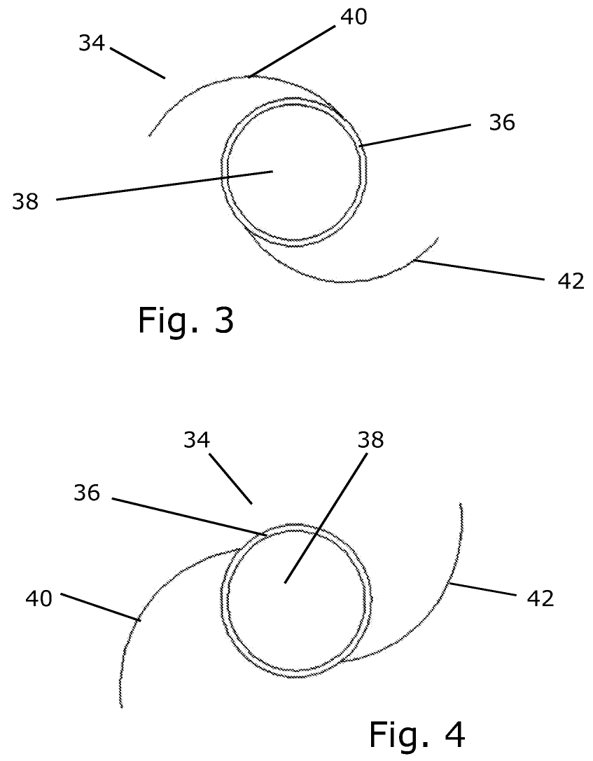

Referring now to FIG. 3, numeral 34 identifies a capsular ring having a circular collar 36 circumscribing and defining a central opening 38. A first, hair-like haptic 40 is attached to collar 36 while a second haptic 42 is attached to collar 36, preferably at a point substantially diametrically across from the point at which haptic 40 is attached. In FIG. 3, haptics 40, 42 are shown as partially deployed such as would be the case when ring 34 has first been inserted into eye 10 and haptics 40, 42 are not yet fully unfolded. In FIG. 4, haptics 40, 42 are shown fully extended.

Use of ring 34 may be best understood by referring now to FIG. 2. After an incision has been made through cornea 12, extending through both epithelium 28 and endothelium 30, ring 34 (which has been previously folded) is inserted through the incision and positioned within anterior chamber 14. It is a well known ophthalmic surgical technique to fold IOLs to decrease the size of the IOL and thereby minimize the size of the incision which must be made through the cornea to allow the IOL to be inserted therethrough. The same technique is used with ring 12 which is formed of a flexible material such as molded silicone, acrylic or polymethyl methacrylate (PMMA) which will allow collar 36 to be folded and haptics 40, 42 to be compressed until ring 34 is inserted and allowed to unfold. When inserted and allowed to unfold, haptics 40, 42 extend to hold ring 34 in place, thereby positioning collar 36 in contact with anterior wall 32 of capsular bag 22.

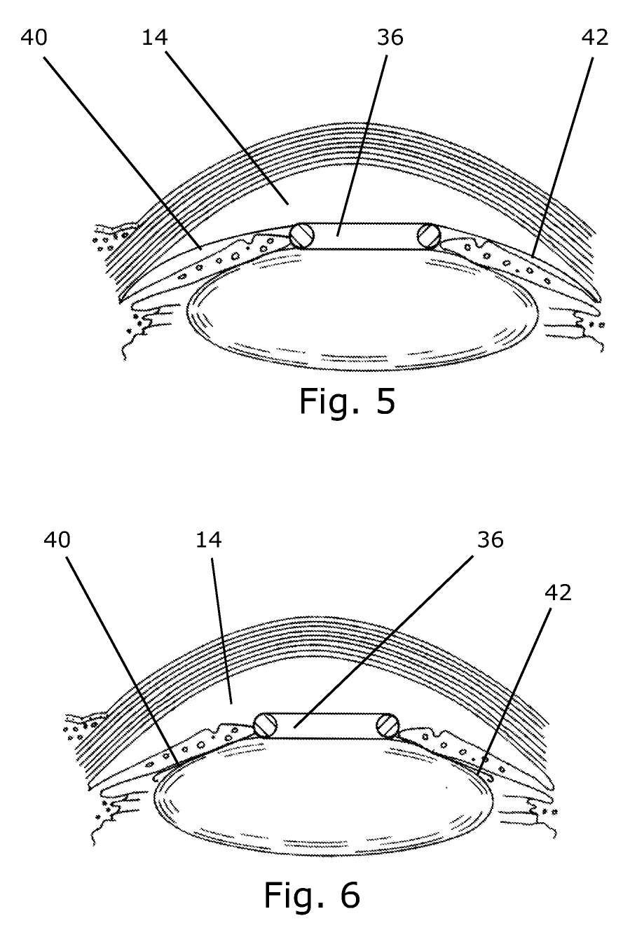

Referring now to FIG. 5 ring 34 is shown installed in anterior chamber 14 with haptics 40, 42 disposed within anterior chamber 14. Alternatively, as shown in FIG. 6, haptics 40, 42 can be tucked under iris 16.

Thereafter, the surgeon will insert a forceps, proceed to make a tear in anterior membrane 32, proximate the inner diameter of collar 36. As the forceps is used to tear membrane 32, collar 36 acts as a guide along which the tear is progressively conducted until a substantially circular section of anterior membrane 32 is successfully removed from capsular bag 22. After capsulorhexis has been completed, ring 34 may be removed through the corneal incision.

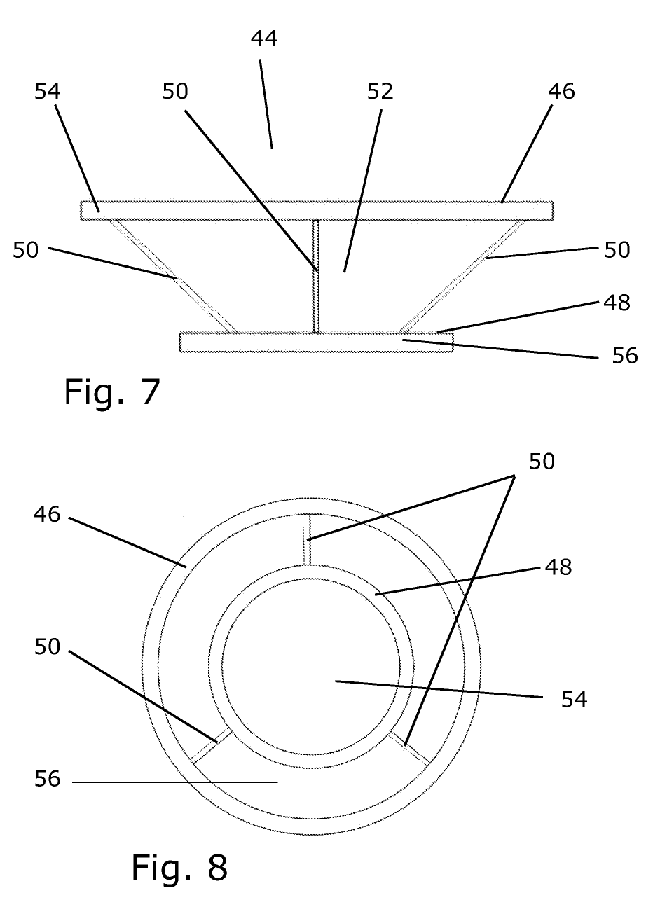

Referring now to FIG. 7, the numeral 44 identifies a capsular guide having an upper circular elastomeric collar 46, a lower elastomeric circular collar 48 and a series of connecting ribs 50 defining a frustoconical space 52. As seen in FIGS. 7 and 8, upper collar 46 has an upper collar opening 54 while lower collar 48 has a lower collar opening 56.

Elastomeric guide 44 is formed from a compressible and foldable material such as molded silicone, acrylic or polymethyl methacrylate (PMMA) which allows guide 44 to be folded and inserted through a relatively small incision made through cornea 12 and thereafter to be distended to fit within anterior chamber 14.

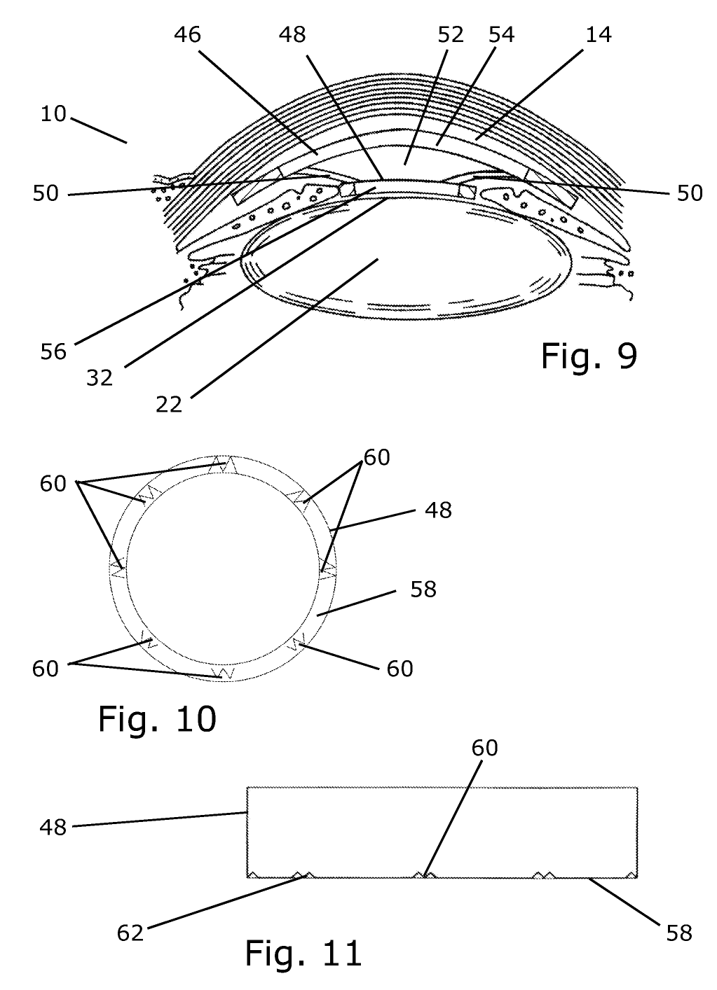

As seen in FIG. 9, when positioned within anterior chamber 14 of eye 10, lower collar 48 is in contact with anterior membrane 32 of capsular bag 22. Ribs 50 are flexed and upper collar 46 is distended to fit within anterior chamber 14 and to hold guide 44 in position. Upper collar opening 54, space 52 and lower collar opening 56 align to allow access to anterior membrane 32 for capsulorhexis to be performed.

With guide 44 thus positioned and held in place by frictional contact within anterior chamber 14, the surgeon may insert a forceps through guide 44 to tear membrane 32 as described above.

Referring now to FIG. 8, upper collar opening 54, space 52 and lower collar opening 56 are shown in a top view demonstrating the space within which the surgeon will work.

Referring now to FIG. 10, the numeral 58 identifies the lowermost surface of lower collar 48 and illustrates formation and placement of a series of grippers 60 spaced about the lower collar surface 58.

As seen in FIG. 11 in a lateral view, each gripper 60 has a projecting point 62 formed into undersurface 58. When positioned within anterior chamber 14 and in contact with capsular bag 22, each gripper 60 will “dig in” to membrane 32, helping to hold guide 44 in place during capsulorhexis. When first brought into contact with capsular bag 22, guide 44 will be slightly rotated by the surgeon to allow each gripper 60 to engage membrane 32.

Once held in position, capsulorhexis may proceed with the surgeon piercing membrane 32 and tearing it in a circular pattern guided by guide 44. After capsulorhexis is completed, guide 44 is then removed.

Another use for ring 34 is to support ciliary zonule 24. To do so, ring 34 is inserted into anterior chamber 14 and is left in place after surgery.

I claim:

1. A guide for conducting capsulorhexis of the capsular bag of an eye, said guide comprising:
a flexible guide ring sized and shaped to fit into the anterior chamber of an eye; and
means for retaining said ring within said anterior chamber during capsulorhexis,
said retaining means configured to allow said guide to be removed from said anterior chamber.

2. The apparatus as recited in claim 1 wherein said retaining means comprises at least one haptic.

3. The apparatus as recited in claim 1 wherein said retaining means is adapted to hold said ring against said capsular bag.

4. A guide for conducting capsulorhexis of the capsular bag of an eye, said guide comprising:
a first, flexible upper ring;
a second, flexible lower ring;
means for attaching said first and second rings together in an opposed, spaced apart configuration,
said attaching means being flexible to allow said first and second rings to be moved one toward another,
at least one of said rings being sized and shaped to fit into the pupil of said eye,
said other of said rings being adapted to retain said guide within said eye.

5. The apparatus as recited in claim 4 wherein one said ring is larger than the other said ring.
